(19) United States
(12) Patent Application Publication
(10) Pub. No.: US 2023/0126241 A1
(43) Pub. Date: Apr. 27, 2023

(54) METHODS AND COMPOSITIONS FOR MULTISTAGE PRIMER EXTENSION REACTIONS

(71) Applicant: Agilent Technologies, Inc., Santa Clara, CA (US)

(72) Inventors: Jinchun Wang, Houston, TX (US); Jurgen Del Favero, Tienen (BE); Weidong Wu, Houston, TX (US); Holly Hogrefe, San Diego, CA (US)

(21) Appl. No.: 17/914,700

(22) PCT Filed: Feb. 3, 2021
(86) PCT No.: PCT/US2021/016307

§ 371 Date: Sep. 26, 2022

Related U.S. Application Data
(60) Provisional application No. 62/994,989, filed on Mar. 26, 2020.

(51) Int. Cl. C12Q 1/686; C12Q 1/6876

(52) U.S. Cl. CPC C12Q 1/686; C12Q 1/6876; C12Q 2600/16

(57) ABSTRACT

Methods and compositions are described for multi-stage primer extension reactions such as multiplex polymerase chain reactions (PCR) and reverse transcriptase PCR. Primer extension stages are performed in a closed vessel without opening the vessel between stages. The multi-stage primer extension methods and compositions utilize earlier stage primers in an earlier stage and later stage primers in a later stage, wherein the later stage primers are blocked from extension during the earlier stage. The blocked primers of the present technology comprise photocleavable blocking groups and are substantially inactive until the blocking group is cleaved by exposure to ultraviolet light. The blocked primers can be activated by ultraviolet light without opening the vessel.

### CROSS-REFERENCE TO RELATED APPLICATIONS

This application is a US national stage entry of PCT/US2021/016307 filed Feb. 3, 2021, which claims the benefit of U.S. Provisional Application No. 62/994,989, filed Mar. 26, 2020, the contents of which are incorporated by reference in their entirety.

### FIELD OF THE INVENTION

The present disclosure is related to methods and compositions for multi-stage primer extension reactions, such as multiplex polymerase chain reaction (PCR) and reverse transcriptase-PCR.

### BACKGROUND

Polymerase Chain Reaction (PCR) is a specific amplification method for DNA sequences. PCR is a useful and widely applied method for DNA target amplification for Next Generation Sequencing (NGS) library preparation. Specifically, primers hybridize to their target sequences in a mixture of nucleic acids and are extended, followed by additional rounds of primer hybridization and extension. PCR enables exponential amplification of the sequence between the primers, making PCR a very sensitive technique. However, PCR can lead to unwanted amplification products. First, if the primers bind other sequences besides their target, off-target amplification can reduce the yield of target sequences. Second, the PCR primers are necessarily at a much higher concentration than the target sequence (to support later rounds of the exponential reaction), and thus, primers can sometimes interact with other primers, creating primer-dimers. Third, different target sequences may amplify with different efficiencies, depending on length, GC content, primer sequence, etc.

Multiplex PCR (mPCR) is a process where many (potentially hundreds or thousands) primers are used in extension reactions. This is convenient, as many target sequences can be amplified in the same tube, and potentially, many target sequences can be amplified from the same aliquot of sample. However, the problems of off-target amplification, primer dimer formation, and uneven amplification are compounded in mPCR. In fact, the primer-dimer problem can become exponentially worse, as each new pair of primers added to the multiplex reaction can potentially interact with all of the other primers in the mixture.

Multiplex PCR has the potential to produce considerable savings of time and effort in the laboratory. The technique has been applied in many areas of DNA testing in humans, including gene deletion analysis, mutation and polymorphism analysis, quantitative analysis, and reverse-transcription (RT)-PCR. In the field of infectious diseases, multiplex PCR has been used for identification of viruses, bacteria, and parasites. The use of mPCR, however, poses several difficulties, including poor sensitivity, poor specificity, preferential amplification of certain specific targets and/or amplification of unintended sequences.

Typically, a multiplex PCR will occur in two separate stages (i.e., multi-stage PCR). In a first stage reaction, target specific primers with universal sequences are used to amplify specific target polynucleotides and add universal forward and reverse sequences onto each amplicon. The products are then purified prior to a later PCR amplification reaction to remove unreacted target specific primers and other reagents. In the later PCR reaction, universal primers designed to hybridize to the universal sequences are then used to amplify the amplicons from the earlier stage and to add any further sequences needed for further processing and identification purposes (such as adapters).

This system is labor intensive. Moreover, the competition between target specific primers and universal primers can result in biased amplification so it is necessary to stop and purify the reaction mixture after the earlier stage. This may introduce possible errors and contamination into the system. Therefore, it would be desirable to identify a method to allow for the performance of both stages in a single reaction vessel. Moreover, it would be particularly advantageous (e.g., for prevention of contamination) and convenient if two or more stages could be performed without opening the reaction vessel.

In reverse transcriptase-polymerase chain reaction (RT-PCR), RNA is reverse transcribed into cDNA in an initial stage, then the cDNA is amplified in a PCR step, usually with target specific primers, in a subsequent stage. RT-PCR suffers the same challenges as multi-step PCR, since gene-specific primers can prime non-specifically during cDNA synthesis which is carried out at relatively low temperature (37-60° C.). Specificity is generally improved by performing reverse transcription and PCR in 2 separate vessels, which prevents the PCR primers from interacting with each other or with the RT primers used to initiate cDNA synthesis, such as oligo(dT), random hexamers, or a gene-specific reverse primer. However, opening tubes between the RT and PCR steps increases labor and the risk of contamination.

### SUMMARY

The present technology is related to novel methods for performing a multi-stage polymerase chain reaction in a closed vessel, wherein the mixture comprises: i) polynucleotide targets, ii) earlier stage primers capable of primer extension, iii) later stage primers comprising a photocleavable blocking group at 3′ ends, iv) primer extension enzyme, and v) other reagents as optionally desired. Examples of earlier stage primers include target specific primers and reverse transcriptase (RT) primers. The target specific primers can comprise a 5′ region and a 3′ region, wherein the 3′ region comprises a target specific sequence, and the 5′ region comprises a universal sequence. When such target specific primers are used in an earlier stage primer extension reaction, the later stage primers can be universal primers which comprise the universal sequence or a portion thereof, and a photocleavable blocking group at their 3′ ends. In some embodiments, the vessel is closed after preparation of the mixture and an earlier stage polymerase chain reaction is performed with the mixture to produce target amplicons. The universal primers are unblocked in the mixture to produce unblocked universal primers comprising the universal sequence or a portion thereof. In some embodiments, the unblocking step is performed without opening the vessel. The later stage primer extension reaction is performed with the unblocked primers and the target amplicons, wherein the unblocked primers amplify the target amplicons. In some embodiments, the unblocking step is performed by exposing the universal primers in the closed vessel to ultraviolet light.

In another aspect, the present technology is related to novel compositions for performing multi-stage PCR wherein the composition comprises a) polynucleotide targets; b) earlier stage primers capable of primer extension, and c) later stage primers comprising a photocleavable blocking group at 3′ ends. Examples of earlier stage primers include target specific primers and reverse transcriptase (RT) primers. The target specific primers can comprise a 5′ region and a 3′ region, wherein the 3′ region comprises a target specific sequence, and the 5′ region comprises a universal sequence. When such target specific primers are the earlier stage primers, the later stage primers can be universal primers which universal primers comprise an universal sequence or a portion thereof, and a photocleavable blocking group at their 3′ ends. In some embodiments the composition is contained in a vessel that is closed after the composition is prepared. The later stage primers can be unblocked and activated for PCR amplification by exposing to ultraviolet light.

In another aspect, the present technology is related to novel methods for performing a multi-stage RT-PCR in a closed vessel, wherein the mixture comprises: i) polyribonucleotide (RNA) targets, ii) earlier stage primers comprising oligo(dT) primers, random primer, or target specific RT primers for cDNA synthesis, iii) later stage primers comprising target specific sequences at their 5′ ends and a photocleavable blocking group at their 3′ ends, iv) a reverse transcriptase, v) a DNA polymerase and vi) other reagents as optionally desired. In some embodiments, the vessel is closed after preparation of the mixture and reverse transcription is performed at a constant temperature (37-60° C.) prior to PCR thermal cycling. The target-specific PCR primers are unblocked without opening the vessel, and the PCR step is performed with the unblocked PCR primers and cDNA, wherein the unblocked primers amplify one or multiple target amplicons. In some embodiments, the unblocking step is performed by exposing the blocked target specific PCR primers in the closed vessel to ultraviolet light.

In another aspect, the present technology is related to novel compositions for performing multi-stage RT-PCR in a closed vessel. The compositions comprises a) polyribonucleotide (RNA) targets; b) earlier stage primers comprising unblocked RT primers for cDNA synthesis; and c) later stage primers comprising blocked primers for a later stage primer extension reaction, wherein the later stage primers comprise a photocleavable blocking group at their 3′ ends. The blocked later stage primers can be target specific PCR primers, random primers, or universal primers. The later stage primers can be unblocked after cDNA synthesis and activated for PCR amplification by exposing to ultraviolet light. In some embodiments, the composition is contained in a vessel that is closed after the composition is prepared.

### BRIEF DESCRIPTION OF THE FIGURES

The skilled artisan will understand that the drawings, described below, are for illustration purposes only. The drawings are not intended to limit the scope of the present teachings in any way.

FIG. 1 provides schematic illustrations of an embodiment of the present multi-stage PCR primers, methods and compositions.

FIG. 2 provides a reverse phase HPLC trace demonstrating the production of a blocked universal primer having a photocleavable blocking group at its 3′ end.

FIG. 3 provides a schematic illustration of an embodiment of the present multi-stage RT-PCR primers, methods and compositions.

FIG. 4 provides a reverse phase HPLC trace demonstrating the unblocking of the present blocked universal primer in PCR tube by exposing the primer to 365 nm ultraviolet light for 10 second.

FIG. 5 provides a Bioanalyzer 2100 image demonstrating that photocleavable blocked primers were only extended in PCR after exposure of the primers to ultraviolet light.

FIGS. 6A and 6B provide BioAnalyzer images of single-vessel RT-PCR reactions carried out with unblocked RT primers in an earlier stage in the presence of blocked later stage primers, which were unblocked for a later stage.

### DETAILED DESCRIPTION

The present technology is related to multi-stage primer extension reactions such as multiplex PCR and RT-PCR using unblocked earlier stage primers and blocked later stage primers. The blocked primers include photocleavable blocking groups at their 3′ ends. Polynucleotide targets are subjected to a primer extension reaction in an earlier stage to form products such as target amplicons or target cDNA. For example, genomic DNA can be amplified in an earlier stage by target specific primers which comprise a target-specific sequence in the 3′ region and a universal sequence in the 5′ region. The product of this earlier stage comprises target amplicons comprising the universal sequences at the 5′ and 3′ ends of the amplicons. The target amplicons are then amplified in a later stage primer extension reaction, by universal primers that have been unblocked by photocleavage of a blocking group, such as by exposure to ultraviolet light. As another example, RNA targets can be subjected to a primer extension in an earlier stage to produce target cDNA. The target cDNA can be subjected to primer extension in a later stage by target-specific primers or universal primers which have been unblocked.

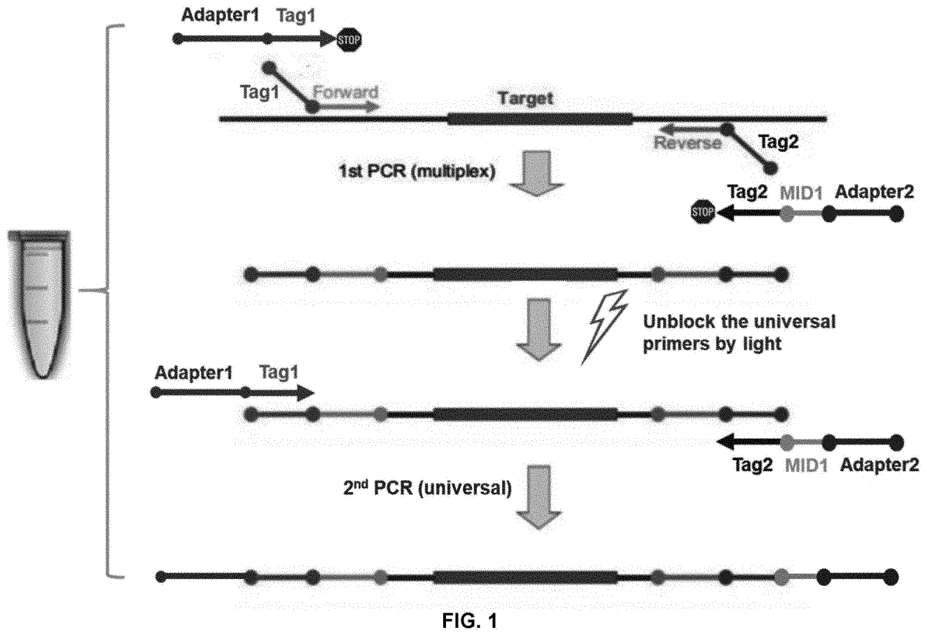

FIG. 1 presents schematic illustrations of a multi-stage PCR approach of the present technology. The PCR approach presented can be performed in a closed vessel containing polynucleotide targets, target specific primers comprising universal sequences, and 3′ blocked photocleavable universal primers comprising photocleavable blocking groups at their 3′ termini.

FIG. 1 shows amplification of a polynucleotide target in an earlier stage PCR by target specific forward and reverse primers. The forward primer comprises a target specific sequence that will hybridize to a target and an universal sequence, indicated as Tag 1, and the reverse primer also comprises a target specific sequence that will hybridize to a target and a different universal sequence, indicated as Tag 2. Amplification of the polynucleotide targets in an earlier stage PCR produces target amplicons comprising polynucleotide target sequences and further comprising universal sequence Tags 1 and 2 (or a complement thereof) at the 5′ and 3′ ends of the target amplicons, respectively. The earlier stage PCR amplification is usually a multiplex PCR amplification where multiple targets are amplified in parallel by multiple sets of target specific primers. Exemplary sets of target specific primers are available from Agilent's SureMASTR technology, such as BRCA MASTR DX Assay.

FIG. 1 also shows a later stage amplification of the target amplicons with a set of 3′ blocked photocleavable universal primers. In the present technology, the forward universal primer shown in FIG. 1 includes a photocleavable blocking group at its 3′ terminus, a 3′ region comprising the sequence of universal sequence Tag 1 or a portion thereof, and an adapter sequence, indicated as Adapter 1, in the 5′ region. The reverse universal primer shown in FIG. 1 includes a photocleavable blocking group at its 3′ terminus, a 3′ region comprising the sequence of universal sequence Tag 2 or a portion thereof, a molecular identifier, indicated as MID 1, and an adapter sequence, indicated as Adapter 2, in the 5′ region. The photocleavable blocking groups in the forward and reverse universal primers are illustrated by Stop signs. The blocked universal primers are present in the reaction mixture during the earlier stage PCR but are substantially inactive with respect to PCR amplification until they are unblocked. The blocked universal primers can be unblocked and activated for the later stage PCR amplification by exposure to ultraviolet light. After exposure to ultraviolet light, the unblocked universal primers are active with respect to PCR amplification. As shown in FIG. 1, a later stage amplification results in universal amplification of the earlier amplicons to produce later stage amplicons. Accordingly, the later stage, universally amplified amplicons comprise the sequences of the target specific primers and the universal primers (e.g., the Adapter 1 and Adapter 2 sequences).

Since the blocked universal primers of the present technology can be unblocked by exposure to ultraviolet light to add the universal primers after the earlier stage of PCR, the universal primers can be present in the mPCR reaction mixture during the earlier stage PCR without interfering with initial target amplification. Surprisingly the blocked universal primers are substantially inactive in the earlier stage but are made active for PCR in a later stage. Also, the photocleavable nature of the blocking groups allows the universal primers to be unblocked and activated by exposing the entire PCR mixture containing vessel to ultraviolet light. This unblocking capability is convenient and highly beneficial, as it reduces or avoids introducing contamination, since later stage PCR can perform without opening the PCR mixture vessel to add the universal primers after the earlier stage PCR.

For example, after the earlier stage of PCR is complete, the 3′ blocked photocleavable universal primers could be unblocked by exposure to ultraviolet light, such as 365 nm light. Exposure to ultraviolet light can be performed in any suitable manner. Unblocking may be performed, for example, by removing the PCR vessel from a thermo-cycler and placing it on an ultraviolet light source such as an ultraviolet light box. Alternatively, a PCR thermo-cycler may be modified to apply the UV light directly. In an event, the ultraviolet light may be applied to unblock the universal primers while they remain in the same, closed PCR reaction vessel. Next, the same, closed PCR reaction vessel can proceed into the later stage of amplification, with the now unblocked universal primers.

In another aspect, the present technology is related to multi-stage RT-PCR where the later stage primers comprise target specific primers that include photocleavable blocking groups at their 3′ ends. Polyribonucleotide targets are reverse transcribed in the first stage by reverse transcriptase and an unblocked RT primer, such as an oligo(dT) primer, a random primer, or a target specific reverse primer. The product of the earlier stage primer extension reaction is target cDNA. The target cDNA can be amplified, during a later stage PCR, by later stage primers, such as target specific primers that have been unblocked by photocleavage of the blocking group.

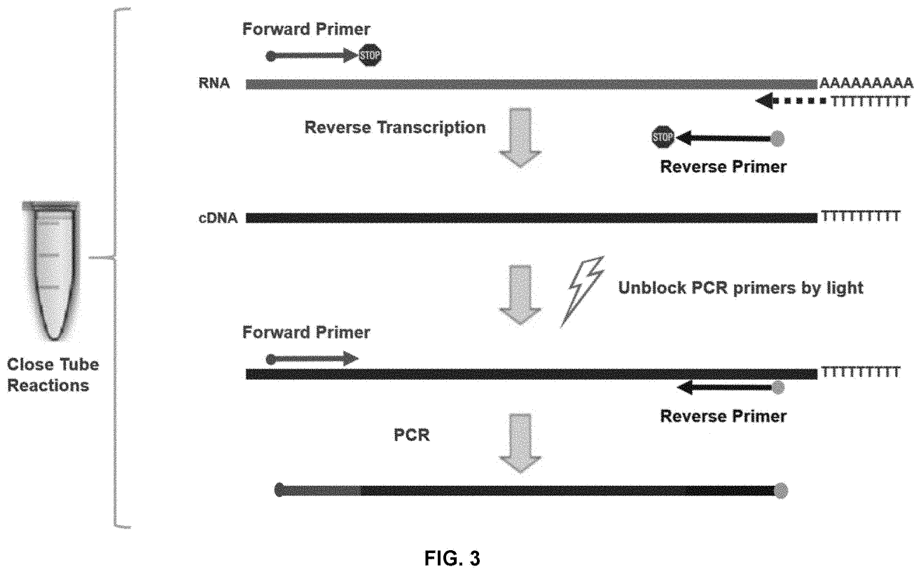

FIG. 3 presents a schematic illustration of a multi-stage RT-PCR of the present technology. The RT-PCR presented can be performed in a closed vessel containing polyribonucleotide targets, an unblocked RT primer, and target specific primers comprising 3′ photocleavable blocking groups. The photocleavable blocking groups in the target specific primers are illustrated by Stop signs. FIG. 3 shows earlier stage reverse transcription of a polyribonucleotide target by unblocked reverse primers in the presence of blocked target specific primers. FIG. 3 also shows later stage amplification of the target amplicons after the 3′ blocking groups have been removed by exposure to ultraviolet light.

In some embodiments, the photocleavable blocking groups of the present technology are connected to a suitable reporter, such as fluorophore. In other embodiments, the photocleavable blocking groups of the present technology are not connected to a reporter. It is contemplated that the photocleavable blocking groups of the present technology will function appropriately whether they include a fluorophore or not, and only require the presence of a photocleavable blocking group.

Also, since the photocleavable blocking groups of the present technology are removed by exposure to ultraviolet light, the present blocking groups do not affect the function of the primer extension enzymes. Accordingly, the photocleavable blocking groups of the present technology are compatible for use with standard PCR and RT-PCR components, such as polymerase enzymes, nucleotides (dNTPs) and buffers.

Before describing exemplary embodiments in further detail, the following definitions and explanations are set forth to illustrate and define the meaning and scope of the terms used in the description.

Numeric ranges are inclusive of the numbers defining the range. Unless otherwise indicated, nucleic acids are written left to right in 5′ to 3′ orientation; amino acid sequences are written left to right in amino to carboxy orientation, respectively.

The present technology may employ, unless otherwise indicated, techniques and descriptions of organic chemistry, polymer technology, molecular biology (including recombinant techniques), cell biology, biochemistry, and immunology, which are within the skill of the art. Such techniques include polymer array synthesis, hybridization, ligation, and detection of hybridization using a label.

As used herein, the singular forms “a”, “an”, and “the” include plural referents unless the context clearly dictates otherwise. For example, the term “a primer” refers to one or more primers, i.e., a single primer and multiple primers. A “plurality” contains at least 2 members. In certain cases, a plurality may have at least 10, at least 100, at least 100, at least 10,000, at least 100,000, at least 10⁶, at least 10⁷, at least 10⁸ or at least 10⁹ or more members.

It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

As used in the specification and appended claims, and in addition to their ordinary meanings, the terms “substantial” or “substantially” mean to within acceptable limits or degree to one having ordinary skill in the art. For example, “substantially inactive” means that one skilled in the art considers the level of activity to be negligible.

The term “sample” as used herein relates to a material or mixture of materials containing one or more polynucleotides or fragments of interest. In some embodiments, the term refers to any plant, animal or viral material containing DNA, RNA, or other polynucleotide, such as, for example, tissue or fluid isolated from a patient (including without limitation plasma, serum, cerebrospinal fluid, lymph, tears, saliva and tissue sections), from preserved tissue (such as FFPE sections) or from in vitro cell culture constituents, as well as samples from the environment. Any sample containing nucleic acid, e.g., genomic DNA from tissue culture cells or from a sample of tissue, may be employed in the present technology.

The term “nucleic acid sample” as used herein denotes a sample containing nucleic acids. The nucleic acid samples may be complex in that they contain multiple different molecules that contain sequences. Nucleic acid samples from a mammal (e.g., mouse or human) are types of complex samples. Complex samples may have more than 10⁴, 10⁵, 10⁶ or 10⁷ different nucleic acid molecules. Also, a complex sample may comprise only a few molecules, where the molecules collectively have more than 10⁴, 10⁵, 10⁶ or 10⁷ or more nucleotides. The term “complexity” generally refers the total number of different sequences in a population, such as in a population of fragments, adapters, or adapter-ligated fragments. For example, if a population has 4 different sequences then that population has a complexity of 4. A population may have a complexity of at least 4, at least 8, at least 16, at least 100, at least 1,000, at least 10,000 or at least 100,000 or more, depending on the desired result.

The term “nucleotide” refers to naturally-occurring nucleotides including guanine, cytosine, adenine, thymine, uracil (G, C, A, T and U respectively), as well as modified pyrimidine and purine derivatives and other non-naturally occurring moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and may contain not only conventional ribose and deoxyribose sugars, but other sugars as well. Modified nucleotides also include modifications on the sugar moiety, e.g., wherein one or more of the hydroxyl groups are replaced with halogen atoms or aliphatic groups, are functionalized as ethers, amines, or the likes.

The term “nucleic acid” and “polynucleotide” are used interchangeably herein to describe a nucleotide-containing polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides or ribonucleotides, and may be produced naturally, chemically, enzymatically or synthetically. The term includes polymers having PNA, LNA or UNA. DNA and RNA have a deoxyribose and ribose sugar backbone, respectively, whereas PNA's backbone is composed of repeating N-(2-aminoethyl)-glycine units linked by peptide bonds. In PNA various purine and pyrimidine bases are linked to the backbone by methylene carbonyl bonds. A locked nucleic acid (LNA), often referred to as inaccessible RNA, is a modified RNA nucleotide. The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired. The term “unstructured nucleic acid”, or “UNA”, is a nucleic acid containing non-natural nucleotides that bind to each other with reduced stability. For example, an unstructured nucleic acid may contain a G′ residue and a C′ residue, where these residues correspond to non-naturally occurring forms, i.e., analogs, of G and C that base pair with each other with reduced stability, but retain an ability to base pair with naturally occurring C and G residues, respectively.

The term “base” refers to a substituted or unsubstituted nitrogen-containing parent heteroaromatic ring of a type that is commonly found in nucleic acids, as well as natural, substituted, modified, or engineered variants or analogs of the same, capable of forming Watson-Crick and/or Hoogsteen hydrogen bonds with an appropriately complementary base.

The term “linker” refers to one or more divalent groups that function as a covalently-bonded molecular bridge between two other groups, such as —C(O)NH—, —C(O)O—, —NH—, —S—, —S(O)n where n is 0, 1 or 2, —O—, —OP(O)(OH)O—, —OP(O)(O⁻)O—, alkanediyl, alkenediyl, alkynediyl, arenediyl, heteroarenediyl, and combinations thereof. Linkers may have pendant side chains or pendant functional groups (or both).

The term “reporter” refers to a chemical moiety that is able to produce a detectable signal directly or indirectly. Examples of reporters include fluorescent dye groups, radioactive labels or groups effecting a signal through chemiluminescent or bioluminescent means. Examples of fluorescent dye groups include zanthene, fluorescein, rhodamine, BODIPY, cyanine, coumarin, pyrene, phthalocyanine, phycobiliprotein, ALEXA FLUOR 350, ALEXA FLUOR 405, ALEXA FLUOR 430, ALEXA FLUOR 488, ALEXA FLUOR 514, ALEXA FLUOR 532, ALEXA FLUOR 546, ALEXA FLUOR 555, ALEXA FLUOR 568, ALEXA FLUOR 568, ALEXA FLUOR 594, ALEXA FLUOR 610, ALEXA FLUOR 633, ALEXA FLUOR 647, ALEXA FLUOR 660, ALEXA FLUOR 680, ALEXA FLUOR 700, ALEXA FLUOR 750, and a squaraine dye. Additional examples, of fluorescent dye reporters that may be used in some embodiments of the present invention are disclosed in Haugland, 2005 and U.S. Pat. Nos. 4,439,356 and 5,188,934, which are incorporated by reference herein. Examples of radioactive labels that may be used as reporters in some embodiments of the present invention, which are well known in the art such as ³⁵S, ³H, ³²P, or ³³P. Examples of reporters that function by chemiluminescent or bioluminescent means and that may be used as reporters in some embodiments of the present invention are described in Nieman, 1989; Given & Schowen, 1989; Orosz et al., 1996; and Hastings, 1983, which are incorporated by reference herein.

The term “oligonucleotide” as used herein denotes a single-stranded multimer of nucleotides generally from about 2 to 200 nucleotides, generally up to 500 nucleotides in length. Oligonucleotides may be synthetic or may be made enzymatically, and, in some embodiments, are 30 to 150 nucleotides in length. Oligonucleotides may contain ribonucleotide monomers (i.e., may be oligoribonucleotides) or deoxyribonucleotide monomers, or both ribonucleotide monomers and deoxyribonucleotide monomers. In some embodiments, the present oligonucleotides may be 10 to 20, 11 to 30, 31 to 40, 41 to 50, 51-60, 61 to 70, 71 to 80, 80 to 100, 100 to 150 or 150 to 200 nucleotides in length, for example.

The term “primer” means an oligonucleotide, either natural or synthetic, that is capable, upon forming a duplex with a polynucleotide template, such as a polynucleotide target, of acting as a point of initiation of nucleic acid synthesis and being extended from its 3′ end along the template so that an extended duplex is formed. The term “extending”, as used herein, refers to the extension of a primer by the addition of nucleotides using a primer extension enzyme. If a primer that is annealed to a nucleic acid is extended, the nucleic acid acts as a template for an extension reaction. The sequence of nucleotides added during the extension process is determined by the sequence of the polynucleotide template. Primers can be extended by a primer extension enzymes such as DNA polymerases and reverse transcriptases. Reverse transcriptases are RNA-dependent DNA polymerases that incorporate deoxynucleotides opposite an RNA template. The resulting cDNA (complementary DNA) can serve as a DNA template in later stage PCR by DNA-dependent DNA polymerases. Primers are generally of a length compatible with their use in synthesis of primer extension products, and are usually are in the range of between 8 to 100 nucleotides in length, such as 10 to 75, 15 to 60, 15 to 40, 18 to 30, 20 to 40, 21 to 50, 22 to 45, 25 to 40, and so on, more typically in the range of between 18-40, 20-35, 21-30 nucleotides long, and any length between the stated ranges. Typical primers can be in the range of between 10-50 nucleotides long, such as 15-45, 18-40, 20-30, 21-25 and so on, and any length between the stated ranges. In some embodiments, the primers are usually not more than about 10, 12, 15, 20, 21, 22, 23, 24, 25, 26, 27, 28, 29, 30, 35, 40, 45, 50, 55, 60, 65, or 70 nucleotides in length.

Primers are usually single-stranded for use in amplification but may alternatively be provided to a mixture in double-stranded form. If double-stranded, the primer is usually first treated to separate its strands before being used to prepare extension products. Thus, a primer is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis. The terms “reverse primer” and “forward primer” refer to primers that hybridize to different strands in a double-stranded DNA molecule, where extension of the primers by a polymerase is in a direction that is towards the other primer. cDNA synthesis can be primed by a reverse transcriptase (RT) primer. For example, an oligonucleotide comprising a series of deoxythymidine nucleotides (oligo(dT)) can be annealed to the 3′ polyA tail of an RNA transcript. Alternatively RT primers can anneal to multiple sequence-specific sites within the RNA (target specific primers). Random primers can also be employed as RT primers.

A “pair” of primers refers to forward and reverse primers designed to amplify a double-stranded polynucleotide target. In some embodiments, the present compositions, methods and kits comprise highly multiplexed sets of target specific primers, for example at least 5 pairs of target specific primers, alternatively at least 10 pairs, or at least 20 pairs, or at least 50 pairs, or at least 100 pairs, or at least 200 pairs, or at least 500 pairs, or at least 1,000 pairs, or at least 2,000 pairs, or at least 5,000 pairs, or at least 10,000 pairs, or at least 20,000 pairs, or more.

The term “primer extension reagents” refers to any reagents that are required or suitable for performing a primer extension reaction (such as a polymerase chain reaction (PCR)) on a polynucleotide molecule such as a polynucleotide target. Primer extension reagents generally include primers, a thermostable polymerase or reverse transcriptase, and nucleotides in a mixture with appropriate buffers. Depending on the enzyme used, ions (e.g., Mg²⁺) may also be present. cDNA synthesis is primed by a reverse primer, annealed to the 3′ polyA tail of an RNA transcript (oligo(dT)), or to multiple sequence-specific sites within the RNA (randomers, target specific primers).

As used herein, the term “universal sequence” refers to a sequence that is common to two or more nucleic acid molecules in a set or population, preferably to substantially all of the nucleic acid molecules in a set or population, where the nucleic molecules also have portions that differ from each other (such as the target portion in a set of polynucleotide target amplicons). A universal sequence can be present in different members of a set or population of molecules, thereby allowing common processing of the different molecules. Non-limiting examples of universal sequences include sequences that are identical to or complementary to the capture sequences of a flow cell. Similarly, a universal sequence can allow the amplification of multiple different nucleic acids using a population of universal amplification primers that are complementary to a portion of the universal sequence, e.g., a universal primer binding site.

In some embodiments, the presently described target specific primers have 5′ regions comprising universal sequences, so that the universal sequences or complements thereof can be incorporated into the target amplicons produced in the earlier PCR stage. Subsequent amplification in later PCR stages using blocked universal primers that have been unblocked and hybridize to the universal sequence can be used to universally amplify the target amplicons present in the PCR mixture.

The terms “upstream” and “5′-of” with reference to positions in a nucleic acid sequence are used interchangeably to refer to a relative position in the nucleic acid sequence that is further towards the 5′ end of the sequence. The terms “downstream” and “3′-of” with reference to positions in a nucleic acid sequence are used interchangeably to refer to a relative position in the nucleic acid sequence that is further towards the 3′ end of the sequence.

As used herein, the term “hybridizing” or “hybridization” refers to any process by which a strand of nucleic acid binds with a complementary strand through base pairing. The terms “hybridize”, “hybridization” also encompass a process in which a nucleic acid strand anneals to and forms a stable duplex, either a homoduplex or a heteroduplex, under normal hybridization conditions with a second complementary nucleic acid strand and does not form a stable duplex with unrelated nucleic acid molecules under the same normal hybridization conditions. The term “duplex,” or “duplexed,” as used herein, describes two complementary polynucleotides that are base-paired, i.e., hybridized together. The formation of a duplex is accomplished by annealing two complementary nucleic acid strands in a hybridization reaction. The hybridization process can be made to be highly specific by adjustment of the hybridization conditions (often referred to as hybridization stringency) under which the hybridization reaction takes place, such that hybridization between two nucleic acid strands will not form a stable duplex, unless the two nucleic acid strands contain a certain number of nucleotides in specific sequences which are substantially or completely complementary. “Normal hybridization” or “normal stringency conditions” are readily determined for any given hybridization reaction.

The term “complementary” refers to two nucleic acids that hybridize with one another under high stringency conditions. The term “perfectly complementary” refers to a duplex in which each base of one of the nucleic acids base pairs with a complementary nucleotide in the other nucleic acid. In many cases, two sequences that are complementary have at least 10, e.g., at least 12 or 15 nucleotides of complementarity. In contrast, if two nucleic acids are “not complementary”, they do not hybridize with one another, though some sequence matches, i.e. a degree of non-complementary less than 100%, may be tolerated so long as the two strands remain in single-stranded form under conditions used in the present methods and as defined above.

The term “amplifying” refers to the process of synthesizing nucleic acid molecules that are complementary to one or both strands of a template nucleic acid such as a polynucleotide target. Amplifying a nucleic acid molecule may include denaturing the template nucleic acid, annealing primers to the template nucleic acid at a temperature that is below the melting temperatures of the primers, and enzymatically elongating from the primers to generate an amplification product. The denaturing, annealing and elongating steps each can be performed one or more times. In certain cases, the denaturing, annealing and elongating steps are performed multiple times such that the amount of amplification product is increasing, often times exponentially, although exponential amplification is not required by the present methods. Amplification typically requires the presence of deoxyribonucleoside triphosphates, a DNA polymerase enzyme and an appropriate buffer and/or co-factors for optimal activity of the polymerase enzyme. The term “amplification product” or “amplicon” refers to the nucleic acid sequences, which are produced from the amplifying process as defined herein. Reverse transcription is a linear amplification reaction that employs a specialized DNA polymerase (reverse transcriptase) to copy RNA into cDNA (complementary DNA) using deoxyribonucleoside triphosphates. When RT-PCR is performed in a single vessel, the buffer and co-factors must support optimal activity of both the reverse transcriptase and the PCR enzyme.

The term “identifier” refers to a sequence of nucleotides can be used to a) identify and/or track the source of a polynucleotide in a reaction, b) count how many times an initial molecule is sequenced and c) pair sequence reads from different strands of the same molecule.

The term “adapter” refers to a nucleic acid attached to polynucleotide, a polynucleotide target or a target amplicon, in preparation for sequencing. The adapter can be attached by primer extension, ligation, or other technique. An adapter can be single stranded or double stranded, and it can comprise DNA, RNA, and/or artificial nucleotides. An adapter can be located at an end of a polynucleotide or it can be located in a middle or interior portion. The adapter can add one or more functional regions to the polynucleotide, such as providing a primer binding site for a later stage primer extension stage or for sequencing, or providing an identifier. By way of example, adapters can include a universal primer and/or a universal priming site, including a priming site for sequencing, and/or a capture site for a NGS sequencing system.

The term “polynucleotide target” refers to a polynucleotide of interest. An isolated polynucleotide target molecule refers to a single molecule that is present in a composition that does not contain other polynucleotide target molecules.

The term “region” refers to a sequence of nucleotides that can be single-stranded or double-stranded.

Other definitions of terms may appear throughout or be understood from the specification.

The precise nucleotide sequences of the target specific primers and the universal primers are generally not critical to the present technology and may be selected by the user based on the teachings of the present disclosure.

Unless defined otherwise, technical and scientific terms used herein have the same meaning as commonly understood by one of ordinary skill in the art to which this disclosure belongs.

All patents and publications, including all sequences disclosed within such patents and publications, referred to herein are expressly incorporated by reference.

As one aspect, the present disclosure provides a method for performing a multi-stage primer-extension reaction in a closed vessel by preparing a primer extension mixture in a vessel, wherein the mixture comprises: i) polynucleotide targets, ii) unblocked primers and iii) blocked primers. The mixture will generally include other primer extension reagents such as deoxyribonucleotide triphosphates (dNTPs), DNA polymerase or other primer extension enzymes, and buffer. In some embodiments, the unblocked primers or the blocked primers comprise target specific primers. The target specific primers comprise a 5′ region and a 3′ region, wherein the 3′ region comprises a target specific sequence, and the 5′ region comprises a universal sequence. The universal primers comprise the universal sequence or a complementary sequence thereof, and a photocleavable blocking group at their 3′ termini. In some embodiments, the vessel is closed after preparation of the mixture and an earlier stage polymerase chain reaction is performed with the mixture to produce target amplicons or target cDNA. The blocked primers can be unblocked in the mixture to produce unblocked primers comprising the universal sequence or a complementary sequence thereof. In some embodiments, the unblocking is performed without opening the vessel. The later stage primer extension reaction can be performed with the unblocked primers and the target amplicons or target cDNA, wherein the unblocked primers amplify the target amplicons. In some embodiments, unblocking and/or the later stage primer extension reactions are performed by photocleaving the blocking groups from the blocked universal primers, such as by exposing the blocked primers in the closed vessel to ultraviolet light.

In additional aspects, the present technology is related to novel compositions for performing primer extension reactions wherein the composition comprises a) polynucleotide targets; b) unblocked primers for an earlier stage and c) blocked primers for a later stage. In some aspects, the target specific primers comprise a 5′ region and a 3′ region, wherein the 3′ region comprises a target specific sequence, and the 5′ region comprises a universal sequence. In other aspects, the universal primers comprise the universal sequence or a portion thereof, and a photocleavable blocking group. In some aspects the composition is prepared in a vessel that is closed after the composition is prepared. In other aspects, the universal primers are unblocked by exposure to ultraviolet light or other photocleavage technique.

In other aspects, the present technology is related to a kit for practicing the present methods, as described above. In some embodiments, the kit may comprise compositions for multi-stage primer extension reactions as described above. In some embodiments, the kit may comprise a mixture comprising unblocked primers such as target specific primers or reverse transcriptase primers, and blocked primers such as universal primers or target specific primers. In some embodiments, the kit comprises a vessel containing a mixture of unblocked target specific primers and blocked universal primers.

In some embodiments, the kit comprises a vessel containing a mixture of unblocked RT primers and blocked target specific primers.

In some embodiments of the present methods and compositions, the earlier stage primers are present at a concentration in the range of 0.01 to 0.5 μM, and the later stage primers are present at a concentration in the range of 0.2 to 1 μM. In some embodiments for multiplex PCR, the target specific primers are present at a concentration in the range of 0.01 to 0.5 μM, and the universal primers are present at a concentration in the range of 0.2 to 1 μM. In some embodiments for RT-PCR, the RT primers are present at a concentration in the range of 0.01 to 0.5 μM, and blocked target specific primers are present at a concentration in the range of 0.2 to 1 μM.

The compositions, methods and kits can be employed to perform multi-stage primer extension reactions on polynucleotide targets such as genomic DNA; mitochondrial DNA, messenger RNA, micro RNA. The polynucleotides targets can be obtained from virtually any organism, including, but not limited to, plants, animals (e.g., reptiles, mammals, insects, worms, fish, etc.), tissue samples, bacteria, fungi (e.g., yeast), phage, viruses, cadaveric tissue, archaeological/ancient samples, etc. In some embodiments, the sample may contain polynucleotide targets from a mammalian cell, such as, a human, mouse, rat, or monkey cell. The sample may be obtained from cultured cells or cells of a clinical sample (e.g., a tissue biopsy, scrape or lavage) or cells of a forensic sample (e.g., cells of a sample collected at a crime scene). In some embodiments, the polynucleotide targets may be obtained from a biological sample such as cells, tissues, bodily fluids, and stool. Bodily fluids of interest include but are not limited to, blood, serum, plasma, saliva, mucous, phlegm, cerebral spinal fluid, pleural fluid, tears, lactal duct fluid, lymph, sputum, synovial fluid, urine, amniotic fluid, and semen. In particular embodiments, the bodily fluid may be obtained from a subject, e.g., a human.

In some embodiments, the polynucleotide targets comprise DNA or RNA obtained from a clinical sample, e.g., a patient that has or is suspected of having a disease or condition such as a cancer, inflammatory disease or pregnancy. In some embodiments, the sample may be made by extracting polynucleotide targets from an archived patient sample, e.g., a formalin-fixed paraffin embedded tissue sample. In some embodiments, the patient sample may be a sample of cell-free circulating DNA from a bodily fluid, e.g., peripheral blood. In some embodiments, the polynucleotide targets used in the earlier stage of the present method is non-amplified DNA that has not been denatured beforehand. In other embodiments, the polynucleotide target in the sample may already be partially fragmented (e.g., as is the case for FFPE samples and circulating cell-free DNA (cfDNA), e.g., ctDNA). In some embodiments, the compositions, methods and kits can be employed to perform multi-stage RT-PCR on polynucleotide targets from RNA, including polyA-fractionated mRNA, from virtually any organism or sample type.

### Later Stage Primers Comprising Blocked 3′ Ends

In some embodiments, the later stage primer is a compound according to Formula I:

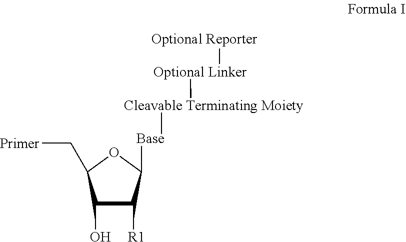

wherein R1 is H or OH.

The Base in Formula I is cytosine, uracil, thymine, adenine, or guanine, or modified pyrimidine and purine derivatives thereof. The Base can be any substituted or unsubstituted nitrogen-containing parent heteroaromatic ring of a type that is commonly found in nucleic acids, as well as natural, substituted, modified, or engineered variants or analogs of the same, capable of forming Watson-Crick and/or Hoogsteen hydrogen bonds with an appropriately complementary base.

The Cleavable Terminating Moiety in Formula I is a group imparting polymerase termination properties to the compound. In some embodiments, the Cleavable Terminating Moiety is a moiety according to the formula:

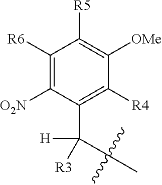

wherein R3 is alkyl(C≤8) or substituted alkyl(C1-8); R4 is hydrogen, hydroxy, halo, amino, nitro, cyano, azido or mercapto; alkyl(C≤6), acyl(C≤6), alkoxy(C≤6), acyloxy(C≤6), alkylamino(C≤6), dialkyl-amino(C≤6), amido(C≤6), or a substituted version of any of these groups; R5 and R6 are each independently: hydrogen, hydroxy, halo, amino, nitro, cyano, azido or mercapto; alkyl(C≤6), alkenyl(C≤6), alkynyl(C≤6), aryl(C≤6), aralkyl(C≤8), heteroaryl(C≤6), acyl(C≤6), alkoxy(C≤6), acyloxy(C≤6), alkylamino(C≤6), dialkylamino(C≤6), amido(C≤6), or a substituted version of any of these groups; a group of formula:

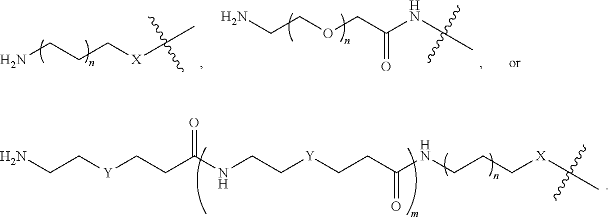

wherein X is —O—, —S—, or —NH—; or alkanediyl(C≤12), alkenediyl(C≤12), alkynediyl(C≤12), or a substituted version of any of these groups; Y is —O—, —NH—, alkanediyl(C≤12) or substituted alkanediyl(C≤12); n is an integer from 0-6; and m is an integer from 0-6; or a -linker-reporter; or a salt, tautomer, or optical isomer thereof.

The Optional Linker in Formula I is one or more divalent groups that function as a covalently-bonded molecular bridge between two other groups, such as —C(O)NH—, —C(O)O—, —NH—, —S—, —S(O)n where n is 0, 1 or 2, —O—, —OP(O)(OH)O—, —OP(O)(O⁻)O—, alkanediyl, alkenediyl, alkynediyl, arenediyl, heteroarenediyl, and combinations thereof. Some linkers have pendant side chains or pendant functional groups (or both). The Optional Reporter is a chemical moiety that is able to produce a detectable signal directly or indirectly. Examples of reporters include fluorescent dye groups, radioactive labels or groups effecting a signal through chemiluminescent or bioluminescent means. In some embodiments, the reporter is selected from the group consisting of xanthene, fluorescein, rhodamine, BODIPY, cyanine, coumarin, pyrene, phthalocyanine, phycobiliprotein, and derivatives thereof.

The Primer in Formula I is an oligonucleotide capable of forming a duplex with a polynucleotide target. In some embodiments, the primer is 8 to 100 nucleotides in length, alternatively 10 to 75, 15 to 60, 15 to 40, 18 to 30, 20 to 40, 21 to 50, 22 to 45, or 25 to 40 nucleotides in length, or another a length within another range disclosed herein.

In some embodiments, the universal primer comprises a 3′ terminal nucleotide selected from the group consisting of (a) 5-[(S)-1-(5-methoxy-2-nitrophenyl)-2,2-dimethyl-propyloxy]methyl-2′-deoxy-uridine, (b) 5-[(S)-1-(5-methoxy-2-nitrophenyl)-2,2-dimethyl-propyloxy]methyl-2′-deoxy-adenosine, (c) 5-[(S)-1-(5-methoxy-2-nitrophenyl)-2,2-dimethyl-propyloxy]methyl-2′-deoxy-guanosine, (d) 5-[(S)-1-(5-methoxy-2-nitrophenyl)-2,2-dimethyl-propyloxy]methyl-2′-deoxy-cytidine, € 5-[(S)-1-(5-methoxy-2-nitrophenyl)-2,2-dimethyl-propyloxy]methyl-2′-deoxy-thymidine, and mixtures thereof, wherein the nucleoside is optionally substituted with a linker and/or a reporter. Exemplary mixtures include mixtures of nucleotides (a) and (b); nucleotides (a), (b) and (c); nucleotides (a), (b), (c) and (d); nucleotides (a), (b), (c), (d) and (e); nucleotides (b) and (c); nucleotides (b), (c) and (d); nucleotides (b), (c), (d) and (e); nucleotides (a) and (c); nucleotides (a) and (d); nucleotides (a) and (e); nucleotides (a), (b) and (d); nucleotides (a), (c) and (d); nucleotides (a), (c), (d) and (e); nucleotides (a), (b), (d) and (e); nucleotides (a), (b), (c) and (e); nucleotides (b) and (d); nucleotides (c) and (d); nucleotides (b) and (e); nucleotides (c) and (e); nucleotides (b), (c) and (e); nucleotides (b), (d) and (e); nucleotides (c), (d) and (e); nucleotides (d) and (e); and any other mixtures.

### Methods, Compositions and Kits for Multiplex and Multi-Stage PCR

As another aspect, the present disclosure provides methods and compositions for improving the efficiency of multiplex nucleic acid amplification. The present disclosure also relates to reagents and methods for improving the efficiency of multi-stage nucleic acid amplification, in particular the performance of two or more amplification reactions designed to occur in sequence in the same reaction mixture or vessel. In particular, composition are provided that have reduced formation of primer dimer and aberrant amplification products. The blocked primers do not form any extendible duplexes before UV deblocking. After the UV deblocking, they become primers capable of primer extension. Such primers are particularly useful where earlier and later amplification reactions take place in a single reaction mixture or vessel. Additional information related to multiplex and multi-stage PCR amplification reactions and reagents related to the same is present in WO2018/10842A1 which is hereby incorporated by reference in its entirety.

In another aspect, the present technology is related to multi-stage RT-PCR using unblocked RT primers as the earlier stage primers, and blocked primers as the later stage primers. For example, the later stage primers can comprise target specific primers comprising photocleavable blocking groups at their 3′ ends. In some embodiments, polyribonucleotide targets are reverse transcribed in the earlier stage by reverse transcriptase with an unblocked RT primer to produce target cDNA. Examples of RT primers include oligo(dT) primers, a randomer (N6-Nn, wherein n can be an integer such as 7, 8, 9, or 10), or a target specific RT primer. The target cDNA are then amplified, during a later stage PCR, such as by target specific primers that have been unblocked by photocleavage of the blocking group.

The present technology is particularly concerned with multiplex nucleic acid amplification in which two or more target sequences are amplified in parallel. This is typically achieved by including more than one pair of polynucleotide target specific primers in a single nucleic acid amplification reaction.

The present technology is also concerned with multi-stage nucleic acid amplification in which two or more distinct amplification reactions take place. Typically, an earlier amplification reaction utilizes target specific primers that amplify the polynucleotide target molecules. The target specific primers include a 5′ region and a 3′ region, wherein the 3′ region comprises a target specific sequence and the 5′ region comprises a universal sequence. The universal sequences are incorporated into amplification products as the reaction proceeds. In a later amplification reaction, universal primers comprising the universal sequences or a portion thereof sufficient to hybridize with the complement of universal sequences are used to amplify the amplification products from the earlier amplification.

The present methods will typically comprise multiple primer extension cycles within each stage. For instance, the earlier stage may comprise at least 3, 4, 5, 6, 7, 8, 9, 10 or more primer extension cycles, and/or at most 20, 18, 16, 14, 12, or fewer PCR cycles. Similarly, the later stage may comprise at least 3, 4, 5, 6, 7, 8, 9, 10 or more primer extension cycles and/or at most 20, 18, 16, 14, 12 or fewer primer extension cycles. The present methods can also comprise additional primer extension stages before or after the earlier stage and/or the later stage. For instance, the earlier stage may be preceded by a primer extension stage to provide input polynucleotides in higher quantity for the earlier stage, and the later stage may be followed by a PCR stage to provide output polynucleotides in higher quantity for sequencing or other application.

In some embodiments, this later amplification involves universal primers that incorporate additional sequences as potentially needed for further, downstream, processing and identification purposes. Thus, the universal amplification is governed by the fact that the later amplification is performed independently of the specific target sequence of the initial target molecule that is amplified. The universal amplification relies upon the incorporation into the amplification products from the earlier amplification reaction of additional sequence (the universal sequences as described herein) that can act as primer binding sites in a later amplification. Thus, the primer region of the primers in the later amplification corresponds to the universal sequence. Primers including such primer regions are referred to herein as “universal primers”.

The universal primers of the present technology comprise photocleavable blocking groups at their 3′ terminus. The blocked universal primers are inactive with respect to PCR amplification even if they are present during a PCR amplification stage. Since the 3′ blocking groups of the present technology are photocleavable, they can be removed by exposing the blocked universal primers to ultraviolet light or other photocleavage technique. Ultraviolet light exposure removes the blocking group and produces a universal primer that is active with respect to PCR amplification. Thus, the universal primers of the present technology can be present but blocked and substantially inactive during an earlier stage of target specific PCR amplification and then activated by exposure to ultraviolet light prior to a later stage of universal PCR amplification.

The polynucleotide targets to be amplified by the present technology are generally not limited. Any suitable polynucleotide target molecule may be amplified using the reagents and methods of the present technology. Multiple different polynucleotide target molecules may be targeted. This may involve use of multiple polynucleotide target specific primer pairs. Thus, the term polynucleotide target generally refers to the desired sequence of a nucleic acid molecule to be amplified, whether as part of the initial polynucleotide target molecule present before amplification begins or a polynucleotide target amplicon molecule generated during amplification.

The polynucleotide targets are molecules comprising or derived from a DNA molecule or a RNA molecule. RNA may be obtained from the same sample types as DNA, as discussed above. The RNA may be messenger RNA (mRNA), microRNA (miRNA) etc. In some embodiments, the RNA is reverse transcribed using a reverse transcriptase enzyme to form a complementary DNA (cDNA) molecule that can then be amplified using the present technology.

The target specific primer pairs of the present technology are designed to amplify the polynucleotide targets and generally incorporate universal sequences. The universal sequences do not hybridize with the initial polynucleotide target molecule. This function is provided by the target specific 3′ region of the target specific primer. However, once the universal sequences have been included in an amplification product they (or their complements) can then act as a primer binding site to which the universal primers hybridize in a later amplification step.

According to some embodiments, the later PCR stage for universal amplification may also be used to include one or more adapter sequences in the later amplicons. The adapter sequence may be any suitable sequence for downstream processing. Downstream processing permits the polynucleotide target or amplicons thereof to be detected and/or quantified from the sample. For example, an adapter sequence complementary to an oligonucleotide immobilized on a suitable solid surface allows a sequence incorporating such an adapter to be immobilized. Other applications rely on the adapter hybridizing to an oligonucleotide in a liquid. Adapters may be useful for array based or sequencing based analyses. In some embodiments, the adapter sequence may be any suitable adapter sequence for high-throughput nucleic acid sequencing. Such sequencing is typically and preferably performed using a next generation sequencing (NGS) platform.

In some embodiments, a universal primer further comprises one or more primer binding sites. For instance, a first (or forward) universal primer may comprise a first primer binding site and the second (or reverse) universal primer may comprise a second primer binding site, wherein the first and second primer binding sites are configured to bind to different primers (e.g., the first and second primer binding sites do not have substantially same sequences and are substantially complementary). The first and/or second primer binding sites can be sequencing primer binding sites, a capture primer binding sites, or a combinations thereof. For example, the first universal primer may comprise a first flow cell amplification primer binding site, and the second universal primer may comprise a second flow cell amplification primer binding site. In some embodiments when the first primer binding site is a sequencing primer binding site, the first universal primer further comprises an identifier upstream of the first primer binding site. For instance, the first universal primer may comprise a universal capture sequence upstream of the identifier. In some embodiments when the second primer biding site is a sequencing primer binding site, the second universal primer further comprises an index downstream of the second primer binding site. For instance the second universal primer may comprise a universal capture sequence or a complement thereof downstream of the identifier. In some embodiments when the first universal primer does not comprise an index, a universal capture site is upstream of the first primer binding site.

In some embodiments, the various primers of the present technology may also be used to add one or more identifiers (also referred to as indexes or barcodes) to the amplification products. For some aspects of the present technology concerning the target-specific primers used in the earlier amplification, sample identifiers and/or molecular identifiers are advantageously included in the primers. In particular embodiments, an identifier may have a length in range of from 2 to 36 nucleotides, or from 6 to 30 nucleotides, or from 8 to 20 nucleotides. In some embodiments, an identifier may contain a “degenerate base region” or “DBR”, where the terms “degenerate base region” and “DBR” refers to a type of molecular identifier that has complexity that is sufficient to help one distinguish between fragments to which the DBR has been added.

The term “sample identifier” refers to a type of identifier that can be added to a polynucleotide, where the sequence identifies the source of the polynucleotide (i.e., the sample from which sample the polynucleotide is derived). In use, each sample is tagged with a different sample identifier sequence (e.g., one sequence is appended to each sample, where the different samples are appended to different sequences), and the tagged samples are pooled. After the pooled sample is sequenced, the sample identifier sequence can be used to identify the source of the sequences. The term “molecular identifier” refers to a type of identifier that can be added to a polynucleotide, where the sequence identifies the individual polynucleotide or an amplicon thereof.

In some embodiments, the universal primers comprise first and second universal primers, wherein the first universal primer comprises, in 5′ to 3′ order: (i) a first adapter sequence; (ii) an molecular identifier; (iii) a universal primer region identical (in the 5′ to 3′ direction) to at least a portion of the first universal sequence; and the second universal primer comprises, in 5′ to 3′ order: (i) a second adapter sequence; (ii) a sample identifier; (iii) a universal primer region identical (in the 5′ to 3′ direction) to at least a portion of the second universal sequence.

In some embodiments, the present technology provides methods for performing multiplex and multi-stage PCR reactions in a single reaction mixture. In some embodiments, all of the amplification steps starting with polynucleotide targets in a PCR mixture up to and including generating the relevant amplification products containing universal sequences (i.e., both earlier and later PCR stages) are carried out without the need to separate, remove or add components. In some embodiments, there is no requirement to perform the target specific amplification stage in a mixture free of universal primers, or to add universal primers between the earlier stage and the later stage, or to purify the earlier amplification products prior to the universal amplification. In some embodiments, all of the PCR reagents required for the method (i.e. to generate the further amplification products) are combined before the earlier amplification stage is carried out. Thus, the method may be performed in a single reaction vessel and without opening the vessel after all of the PCR reaction mixture components are added. The reaction vessel does not need to be further manipulated, or opened, once the reaction mixture has formed (apart from performing the amplification itself e.g. thermal cycling) until the universal amplification products have been generated. The present methods may, therefore, be considered to be “closed vessel” methods. The present methods are highly advantageous in that the user does not need to add the universal primers between the earlier and later stages.

In some embodiments, all of the primer extension stages, starting with polynucleotide targets in a mixture up to and including generating the relevant target amplification products containing universal sequences (i.e., both earlier and later stage primer extension stages) are carried out without the need to separate, remove or add components. In some embodiments, there is no requirement to perform the target specific amplification stage in a mixture free of universal primers, or to add universal primers between the earlier stage and the later stage, or to purify the earlier target amplification products prior to the universal amplification. In some embodiments, all of the primer extension reagents required for the method (i.e. to generate the further amplification products) are combined before the earlier amplification stage is carried out. Thus, the present methods may be performed in a single reaction vessel and without opening the vessel after all of the components of the reaction mixture components are added. The reaction vessel does not need to be further manipulated, or opened, once the reaction mixture has formed (apart from performing the amplification itself e.g. thermal cycling) until the universal amplification products have been generated. The present methods may, therefore, be considered to be “closed vessel” methods. The present methods are highly advantageous in that the user does not need to add the later stage primers between the earlier and later stages.

The present methods also encompass the performance of additional steps after the generation of the universal amplification products (i.e. after the universal, or later stage, amplification). Such methods are not constrained to the same reaction mixture or reaction vessel. Such methods may involve detecting optionally quantifying the polynucleotide target molecule. In some embodiments, the methods of the present technology are used to identify and optionally quantify specific polynucleotide target molecules. In other embodiments the method further comprises sequencing the further amplification products. Sequencing is typically performed in massively parallel fashion such as by using a next generation sequencing (NGS) technique. The sequencing may take place in a different reaction mixture to that of the amplification reactions of the present technology.

The present technology is also concerned with multi-stage RT-PCR reactions in which two or more distinct amplification reactions take place in a single vessel. cDNA synthesis is performed independently of PCR by blocking the 3′ ends of target specific PCR primers with a photocleavable blocking group. cDNA synthesis is performed at a constant temperature that is optimal for reverse transcriptase, without interference from PCR primers that would otherwise interact non-specifically to produce primer-dimers and other artifacts. Thus, the target specific PCR primers of the present technology can be present but substantially inactive during cDNA synthesis, and then activated by exposure to ultraviolet light prior to a later stage primer extension reaction (such as PCR). As described above for multi-stage mPCR reactions, the RT-PCR method can be performed in a single vessel with no further manipulations.

Also provided by this disclosure are kits for practicing the present methods, as described herein. In some embodiments, the kit may comprise compositions for multi-stage PCR as described above. In some embodiments, the kit may comprise a PCR mixture comprising target specific primers and blocked universal primers having a photocleavable blocking group at their 3′ terminus. The target specific primers and blocked universal primers may be in a mixture in a single vessel. The kits of the present technology may additionally comprise suitable reagents (e.g., buffers etc.) for performing a multi-stage PCR. The various components of the kit may be present in separate containers or certain compatible components may be precombined into a single container, as desired. In addition to the reagents described above, a kit may contain any of the additional components used in the method described above, e.g., one or more enzymes and/or buffers, etc.

In some embodiments, the kit may comprise compositions for multi-stage RT-PCR as described above. In some embodiments, the kit may comprise an RT-PCR mixture comprising cDNA synthesis primers (oligo(dT) or randomers) and target specific primers having a photocleavable blocking group at their 3′ terminus. The cDNA synthesis and blocked target specific primers may be in a mixture in a single vessel. The kits of the present technology may additionally comprise suitable reagents for performing a multi-stage RT-PCR, which may be provided in any format described above.

In addition to above-mentioned components, the kits may further include instructions for using the components of the kit to practice the present methods, i.e., to instructions for multi-stage amplification of polynucleotide targets. The instructions for practicing the present methods can be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

### Photocleavable Blocking Groups

The present technology involves primers that are reversibly blocked with photocleavable blocking groups at the 3′ terminus. These blocked primers can be present during an earlier stage primer extension reaction but are blocked from extension during that stage. After they are unblocked, they become capable of extension in a later stage primer extension reaction. The photocleavable blocking groups of the present technology include nucleotides that are attached to the 3′ end of the later stage primers where they block PCR amplification. The blocked primers are substantially inactive with respect to PCR amplification until they are unblocked and activated by exposure to ultraviolet light or other photocleavage technique. A wide variety of photocleavable blocking groups can be included in the later stage primers, such as those described in U.S. Pat. Nos. 8,969,535, 9,200,319, and 10,041,115 which are incorporated herein by reference in their entireties. It is contemplated that the present later stage primer can comprise any photocleavable blocking group at its 3′ end such that the later stage primer is substantially inactive with respect to PCR amplification until they are unblocked. In some embodiments, the photocleavable blocking group has a blocking efficiency from about 90% to about 100%.

The photocleavable blocking groups are designed to reversibly block and terminate DNA synthesis, and then be cleaved efficiently by exposure to ultraviolet light, thereby actuating the primer. In some embodiments, the photocleavable blocking groups are in the form of nucleotide compounds containing the bases adenine, cytosine, guanine, thymine, uracil, or modified pyrimidine and purine derivatives thereof such as 7-Hydroxyl-7-deaza-adenine/guanine. In other embodiments, the cleavable groups can be derivatized to include a reporter such as a dye. In some embodiments, the bases adenine, cytosine, guanine, thymine, uracil, or modified pyrimidine and purine derivatives thereof, can be covalently attached to a photocleavable protecting group such as a 2-nitrobenzyl group. In some embodiments, the 2-nitrobenzyl group is derivatized to enhance its termination of DNA synthesis. The photocleavable protecting group, such as the 2-nitrobenzyl group, also can be derivatized, in some embodiments, with a fluorescent dye by covalent linkage to the photocleavable protecting group.

In some embodiments, the photocleavable blocking groups comprise the base of the nucleoside covalently attached with a 2-nitrobenzyl group, and the alpha carbon position of the 2-nitrobenzyl group is optionally substituted with one alkyl or aryl group. In other embodiments, the 2-nitrobenzyl group is functionalized to enhance the termination and blocking properties as well as the light catalyzed deprotection rate. In other embodiments, the termination and blocking properties of the 2-nitrobenzyl and alpha carbon substituted 2-nitrobenzyl group attached to the base occur even when the 3′-OH group on the ribose sugar is unblocked. In some embodiments, the photocleavable blocking groups are selected to be well-tolerated by a number of commercially available DNA polymerases. In some embodiments, the alpha carbon substituted 2-nitrobenzyl group also can be derivatized to include a selected fluorescent dye or other reporter.

### Methods of Preparing Photocleavable Blocking Groups

The photocleavable blocking groups are in the form nucleotide compounds that include photocleavable protecting groups that are designed to terminate DNA synthesis as well as cleave rapidly. They are combined with and added to the 3′ end of a primer precursor, such as by a single-base extension of the primer precursor annealed to a template with a DNA polymerase, or alternatively by a single base extension of the primer precursor in a template-independent manner with a terminal deoxynucleotide transferase (TdT). Accordingly, universal primers comprising the photocleavable blocking groups are inactive for further extension.

In another embodiment, the nucleotide comprising a photocleavable blocking group is a compound according to the formula that can be attached to the 3′ end of a universal primer:

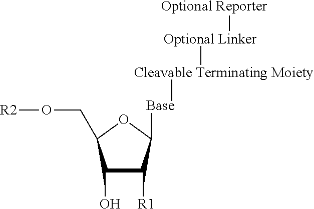

wherein R1 is H or OH, R2 is H, monophosphate, diphosphate, triphosphate, or α-thiotriphosphate, base is cytosine, uracil, thymine, adenine, or guanine, or modified pyrimidine and purine derivatives thereof, cleavable terminating moiety is a group imparting polymerase termination properties to the compound, optional linker is a bifunctional group. The Base in Formula II is cytosine, uracil, thymine, adenine, or guanine, or modified pyrimidine and purine derivatives thereof. As noted above, a Base can be any substituted or unsubstituted nitrogen-containing parent heteroaromatic ring of a type that is commonly found in nucleic acids, as well as natural, substituted, modified, or engineered variants or analogs of the same, capable of forming Watson-Crick and/or Hoogsteen hydrogen bonds with an appropriately complementary base.

The Cleavable Terminating Moiety in Formula II is a group imparting polymerase termination properties to the compound. The Optional Linker in Formula I is one or more divalent groups that function as a covalently-bonded molecular bridge between two other groups. The Optional Reporter is a chemical moiety that is able to produce a detectable signal directly or indirectly. Examples of Cleavable Terminating Moieties, Optional Linkers, and Optional Reporters are set forth above with respect to Formula I, and those exemplary Cleavable Terminating Moieties, Optional Linkers, and Optional Reporters can also be incorporated in Formula II.

### EXAMPLES

### Example 1: Production of Photocleavable 3′ Blocked Primer

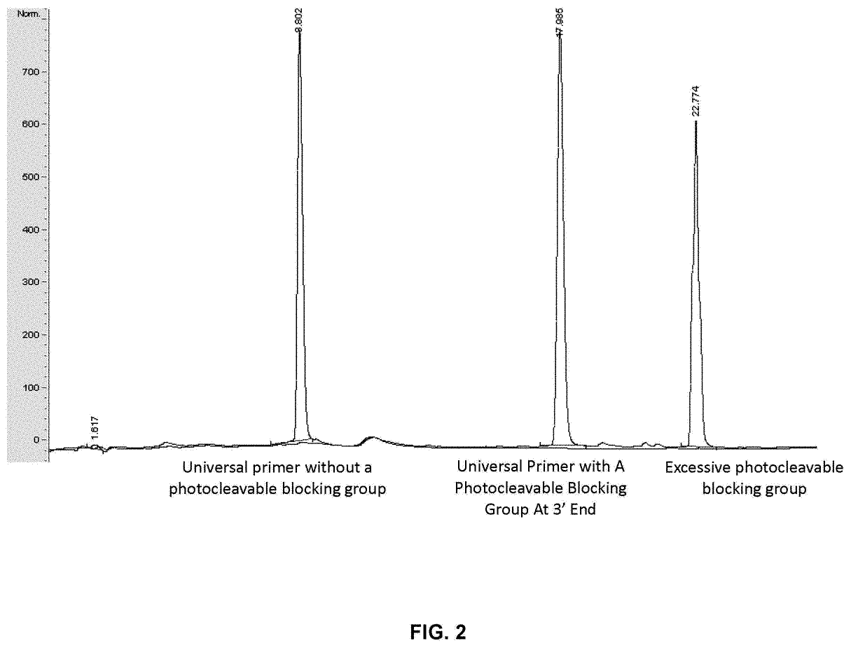

In this example, a primer was synthesized with a blocking group on its 3′ terminus. A photocleavable blocked primer was produced by performing a single-base extension of a primer precursor. A primer precursor (Numb1-1) was annealed to a DNA template and a nucleotide comprising a photocleavable blocking group (LT-dG) was incorporated at the 3′ end of the primer precursor by single-base extension. Products were purified and analyzed by reverse-phase high performance liquid chromatography (HPLC). FIG. 2 shows the product of the annealed primer and template prior to addition of LT-dG as the peak to the far left, the single-base extension product of the primer with LT-dG added as the middle peak and in the peak to the far right, excess and unincorporated LT-dG.

### Example 2: Photocleavable Blocked Primer can be Unblocked by Ultraviolet Light

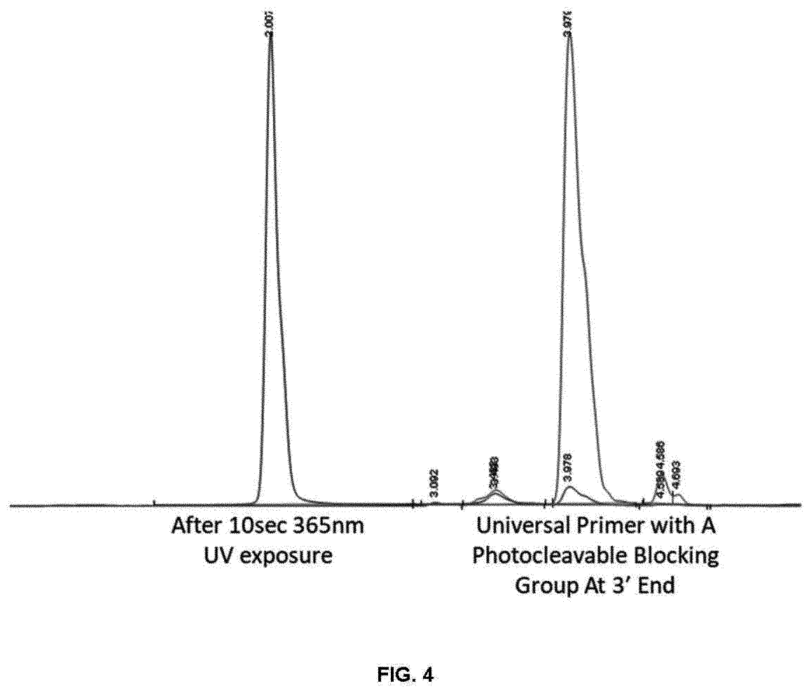

In this example, the ability to unblock a primer having a blocking group at its 3′ terminus was evaluated. FIG. 4 shows an HPLC trace illustrating the HPLC purified universal primer with a photocleavable blocking group at a 3′ end (the major peak on the right) and the universal primer after 10 seconds of 365 nm UV light exposure (the peak on the left). The increased HPLC mobility is due to cleavage of the photocleavable blocking group from the 3′ end of the primer by ultraviolet light. This demonstrates that the photocleavable blocked primers of the present technology can be efficiently unblocked by ultraviolet light and are then competent to be extended by a DNA polymerase.

### Example 3: Photocleavable Blocked Primers can be Extended in PCR Only after Exposure to Ultraviolet Light

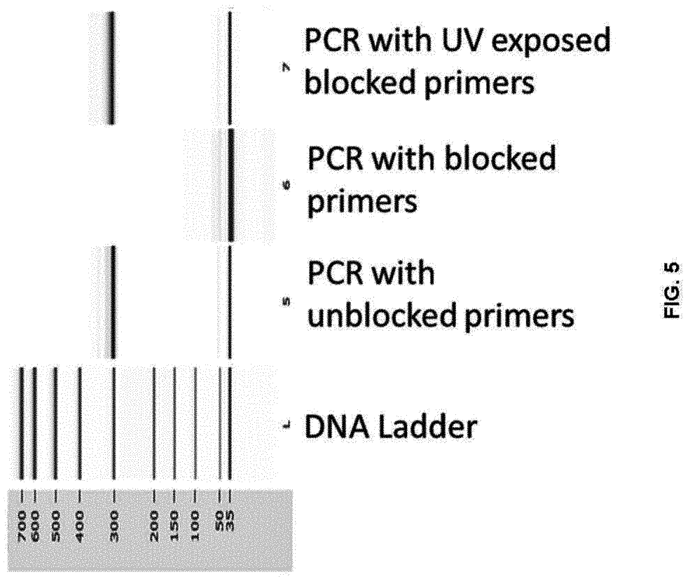

In this example, the use of the blocked primers for PCR amplification was evaluated. FIG. 5 shows a Bioanalyzer 2100 image of three PCR products. The lane marked “PCR with unblocked primers” is for a positive control, showing amplification products of a 305 bp fragment of gDNA with unblocked primers (Numb1-FP and Numb1-RP). The lane marked “PCR with blocked primers” is for PCR attempted with an unblocked reverse primer (Numb1-RP) and a photocleavable blocked forward primer (Numb1-F*). This lane exhibits minimal PCR amplification, due to blocked primer not being able to be extended PCR amplification. The lane marked “PCR with UV exposed blocked primers” is for PCR with the unblocked reverse primer and the photocleavable blocked forward primer, after the blocking group on the forward primer (Numb1-F*) was cleaved by exposure to ultraviolet light. This lane shows amplification of the PCR product since the forward primer was unblocked and made capable for being extended in PCR amplification. These results demonstrate that the blocked primers of the present technology are not extended in PCR amplification but can be unblocked and activated to be extended PCR amplification by exposure to ultraviolet light.

### Example 4: Photocleavable Blocked Primers can be Extended in RT-PCR Only after Exposure to Ultraviolet Light

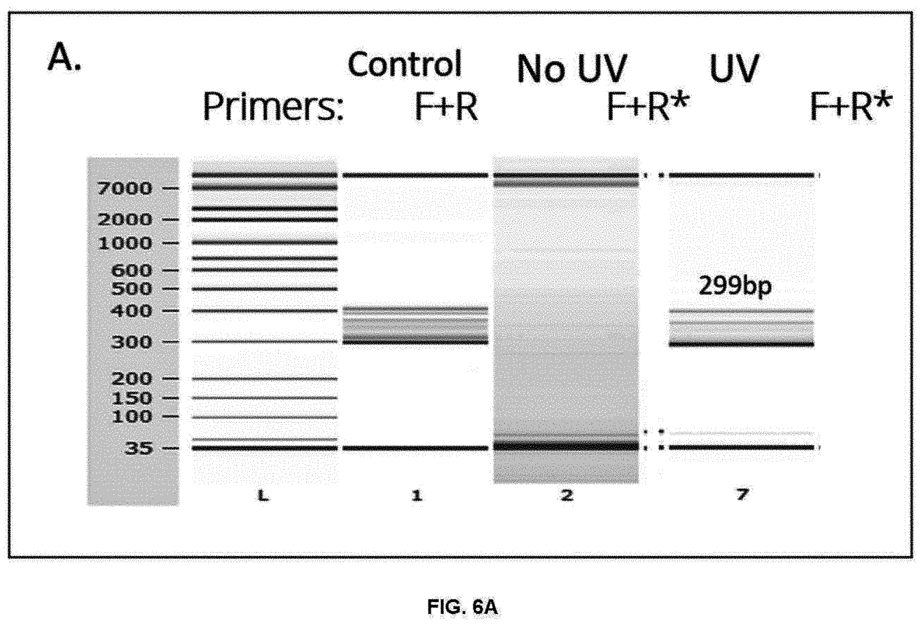

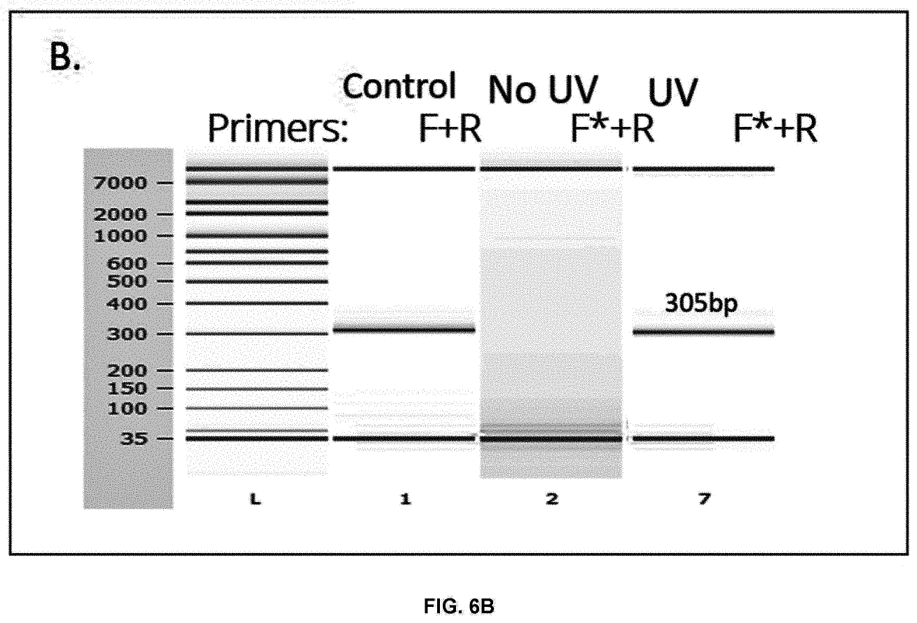

In this example, the use of blocked target specific primers was evaluated in RT-PCR as another embodiment of a multi-stage primer extension reaction. FIG. 6 shows Bioanalyzer 2100 images of products from single-vessel RT-PCR reactions carried out with a photocleavable blocked β-actin reverse primer (Panel A; R*) or a photocleavable blocked Numb1 forward primer (Panel B; F*). Closed tubes were exposed to UV for 3 minutes between cDNA synthesis and thermal cycling. In the absence of UV exposure, no target specific products are generated in either assay, indicating that β-actin R* and Numb1 F* remain inactive during both cDNA synthesis and PCR steps. With β-actin, additional controls showed that reverse transcription was primed from β-actin R* in the period between UV exposure and the initial PCR denaturation step (not shown). Non-specific interactions could be prevented during this short time frame by performing UV exposure at elevated temperature. Results show that the blocked target specific primers are only extended in RT-PCR after exposure to ultraviolet light.

### EXEMPLARY EMBODIMENTS

Exemplary embodiments provided in accordance with the presently disclosed subject matter include, but are not limited to, the following:

Embodiment 1. A method for performing a multi-stage primer extension reaction in a closed vessel. The method comprises a) preparing a primer extension mixture in a vessel, wherein the mixture comprises i) polynucleotide targets; ii) earlier stage primers capable of primer extension; iii) later stage primers comprising a photocleavable blocking group at 3′ ends; iv) primer extension enzyme; and v) primer extension reagents. The vessel is closed after preparation of the mixture. The method also comprises b) performing an earlier stage primer extension reaction with the earlier stage primers to produce target amplicons or target cDNA. The method comprises c) unblocking the later stage primers to produce unblocked later stage primers, wherein the unblocking step is performed without opening the vessel. The method also comprises d) performing a later stage primer extension reaction with the unblocked later stage primers and the target amplicons or target cDNA. The unblocked later stage primers hybridize to the target amplicons or target cDNA and are extended.

Embodiment 2. The method of embodiment 1, wherein the earlier stage primers comprise target specific primers comprising a 5′ region and a 3′ region, wherein the 3′ region comprises a target specific sequence, and the 5′ region comprises a universal sequence.

Embodiment 3. The method of embodiment 2, wherein the later stage primers comprise universal primers comprising said universal sequence or a portion thereof.

Embodiment 4. The method of any of embodiments 1 to 3, wherein the earlier stage primers comprise reverse transcriptase (RT) primers.

Embodiment 5. The method of embodiment 4, wherein the later stage primers comprise target specific primers.

Embodiment 6. The method of any of embodiments 1 to 5, wherein the unblocking step (c) comprises exposing the later stage primers in the closed vessel to ultra-violet light.

Embodiment 7. The method of any of embodiments 1 to 6, wherein the blocked primers are compounds according to Formula I:

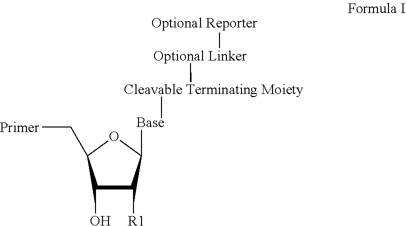

wherein R1 is H or OH; Base is cytosine, uracil, thymine, adenine, or guanine, or modified pyrimidine and purine derivatives thereof; Cleavable Terminating Moiety is a group imparting polymerase termination properties to the compound; Optional Linker is a divalent group; Optional Reporter is a chemical moiety that is able to produce a detectable signal directly or indirectly; and Primer is an oligonucleotide capable of forming a duplex with a polynucleotide target.

Embodiment 8. The method of embodiment 7, wherein the Cleavable Terminating Moiety is a moiety according to the following formula:

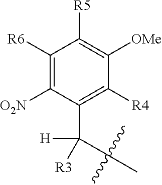

wherein:

R3 is alkyl(C≤8) or substituted alkyl(C1-8);

R4 is hydrogen, hydroxy, halo, amino, nitro, cyano, azido or mercapto; alkyl(C≤6), acyl(C≤6), alkoxy(C≤6), acyloxy(C≤6), alkylamino(C≤6), dialkyl-amino(C≤6), amido(C≤6), or a substituted version of any of these groups;

R5 and R6 are each independently: hydrogen, hydroxy, halo, amino, nitro, cyano, azido or mercapto; alkyl(C≤6), alkenyl(C≤6), alkynyl(C≤6), aryl(C≤6), aralkyl(C≤8), heteroaryl(C≤6), acyl(C≤6), alkoxy(C≤6), acyloxy(C≤6), alkylamino(C≤6), dialkylamino(C≤6), amido(C≤6), or a substituted version of any of these groups; a group of formula:

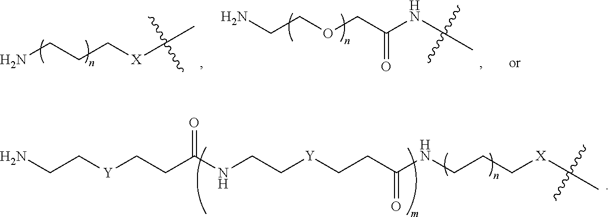

X is —O—, —S—, or —NH—; or alkanediyl(C≤12), alkenediyl(C≤12), alkynediyl(C≤12), or a substituted version of any of these groups;

Y is —O—, —NH—, alkanediyl(C≤12) or substituted alkanediyl(C≤12); n is an integer from 0-6; and

m is an integer from 0-6; or a -linker-reporter;

or a salt, tautomer, or optical isomer thereof.

Embodiment 9. The method of embodiment 7, wherein the Cleavable Terminating Moiety comprises a 2-nitrobenzyl substituent.

Embodiment 10. The method of any of embodiments 7 to 9, wherein the Primer is selected from oligonucleotides having a length between 8 to 100 nucleotides.

Embodiment 11. The method of any of embodiments 7 to 10, wherein the Base is selected from the group consisting of adenine, cytosine, guanine, thymine, uracil, modified pyrimidine and purine derivatives thereof, and mixtures thereof.

Embodiment 12. A composition for performing a multi-stage primer extension reaction comprising a) polynucleotide targets, b) earlier stage primers capable of primer extension; and c) later stage primers comprising a photocleavable blocking group at 3′ ends, and wherein the composition is in a vessel that is closed upon preparation of the composition.

Embodiment 13. The composition of claim 12, wherein the later stage primers are configured for unblocking by exposure to ultraviolet light.

Embodiment 14. The composition of any of embodiments 12 to 13, wherein the photocleavable blocking group has a blocking efficiency from about 90% to about 100%.

Embodiment 15. The composition of any of embodiments 12 to 14, comprising at least 5 pairs of target specific primers, alternatively at least 5 pairs of target specific primers, alternatively at least 10 pairs, or at least 20 pairs, or at least 50 pairs, or at least 100 pairs, or at least 200 pairs, or at least 500 pairs, or at least 1,000 pairs, or at least 2,000 pairs, or at least 5,000 pairs, or at least 10,000 pairs, or at least 20,000 pairs, of target specific primers.

Embodiment 16. The composition of any of embodiments 12 to 15, the earlier stage primers are present at a concentration of 0.01 to 0.5 μM, and the later stage primers are present at a concentration of 0.2 to 1 μM.

Embodiment 17. The composition of any of embodiments 12 to 16, wherein the later stage primers comprise a 3′ terminal nucleotide that is selected from the group consisting of:
5-[(S)-1-(5-methoxy-2-nitrophenyl)-2,2-dimethyl-propyloxy]methyl-2′-deoxy-uridine,
5-[(S)-1-(5-methoxy-2-nitrophenyl)-2,2-dimethyl-propyloxy]methyl-2′-deoxy-adenosine,
5-[(S)-1-(5-methoxy-2-nitrophenyl)-2,2-dimethyl-propyloxy]methyl-2′-deoxy-guanosine,
5-[(S)-1-(5-methoxy-2-nitrophenyl)-2,2-dimethyl-propyloxy]methyl-2′-deoxy-cytidine,
5-[(S)-1-(5-methoxy-2-nitrophenyl)-2,2-dimethyl-propyloxy]methyl-2′-deoxy-thymidine,
   and mixtures thereof, including mixtures of any two, three, four or five of the foregoing nucleotides.

Embodiment 18. A method of preparing a photocleavable blocked primer comprising a) providing a primer precursor having a 3′ end; and b) i) forming a duplex of the primer precursor hybridized to a template, wherein the template has a 5′ overhang relative to at least one nucleotide of the 3′ end of the primer precursor; and extending the primer precursor at its 3′ end by incorporating a nucleotide comprising a photocleavable blocking group with an DNA polymerase; or ii) extending the primer precursor at its 3′ end by incorporating a nucleotide comprising a photocleavable blocking group with an template independent DNA polymerase.

Embodiment 19. The method of claim 18, wherein the nucleotide comprising a photocleavable blocking group is a compound of Formula II:

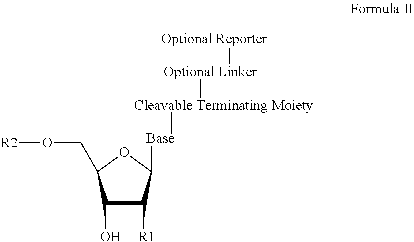

wherein R1 is H or OH; R2 is H, monophosphate, diphosphate, triphosphate or α-thiotriphosphate; Base is cytosine, uracil, thymine, adenine, or guanine, or modified pyrimidine and purine derivatives thereof; Cleavable Terminating Moiety is a group imparting polymerase termination properties to the compound; Optional Linker is a divalent group; and Optional Reporter is a chemical moiety that is able to produce a detectable signal directly or indirectly.

1. A method for performing a multi-stage primer extension reaction in a closed vessel comprising:
a) preparing a primer extension mixture in a vessel, wherein the mixture comprises:
i) polynucleotide targets;
ii) earlier stage primers capable of primer extension;
iii) later stage primers comprising a photocleavable blocking group at 3′ ends;
iv) primer extension enzyme; and
v) primer extension reagents,
wherein the vessel is closed after preparation of the mixture;
b) performing an earlier stage primer extension reaction with the earlier stage primers to produce target amplicons or target cDNA;
c) unblocking the later stage primers to produce unblocked later stage primers, wherein the unblocking step is performed without opening the vessel; and
d) performing a later stage primer extension reaction with the unblocked later stage primers and the target amplicons or target cDNA,
wherein the unblocked later stage primers hybridize to the target amplicons or target cDNA and are extended.

2. The method of claim 1, wherein the earlier stage primers comprise target specific primers comprising a 5′ region and a 3′ region, wherein the 3′ region comprises a target specific sequence, and the 5′ region comprises a universal sequence.

3. The method of claim 2, wherein the later stage primers comprise universal primers comprising said universal sequence or a portion thereof.

4. The method of claim 1, wherein the earlier stage primers comprise reverse transcriptase (RT) primers.

5. The method of claim 4, wherein the later stage primers comprise target specific primers.

6. The method of claim 1, wherein the unblocking step (c) comprises exposing the later stage primers in the closed vessel to ultra-violet light.

7. The method of claim 1, wherein the blocked primers are compounds according to Formula I: 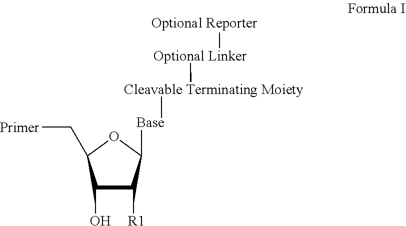
wherein:
R1 is H or OH;
Base is cytosine, uracil, thymine, adenine, or guanine, or modified pyrimidine and purine derivatives thereof;
Cleavable Terminating Moiety is a group imparting polymerase termination properties to the compound;
Optional Linker is a divalent group;
Optional Reporter is a chemical moiety that is able to produce a detectable signal directly or indirectly; and
Primer is an oligonucleotide capable of forming a duplex with a polynucleotide target.

8. The method of claim 7, wherein the Cleavable Terminating Moiety is a moiety according to the following formula: 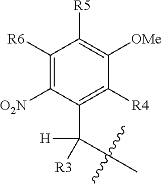
wherein:
R3 is alkyl(C≤8) or substituted alkyl(C1-8);
R4 is hydrogen, hydroxy, halo, amino, nitro, cyano, azido or mercapto; alkyl(C≤6), acyl(C≤6), alkoxy(C≤6), acyloxy(C≤6), alkylamino(C≤6), dialkyl-amino(C≤6), amido(C≤6), or a substituted version of any of these groups;
R5 and R6 are each independently: hydrogen, hydroxy, halo, amino, nitro, cyano, azido or mercapto; alkyl(C≤6), alkenyl(C≤6), alkynyl(C≤6), aryl(C≤6), aralkyl(C≤8), heteroaryl(C≤6), acyl(C≤6), alkoxy(C≤6), acyloxy(C≤6), alkylamino(C≤6), dialkylamino(C≤6), amido(C≤6), or a substituted version of any of these groups; a group of formula: 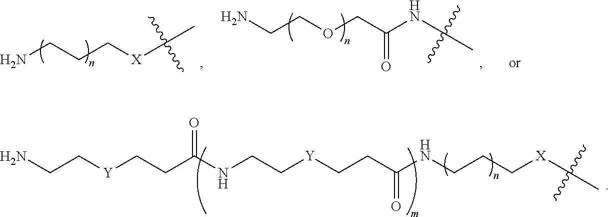
X is —O—, —S—, or —NH—; or alkanediyl(C≤12), alkenediyl(C≤12), alkynediyl(C≤12), or a substituted version of any of these groups;
Y is —O—, —NH—, alkanediyl(C≤12) or substituted alkanediyl(C≤12); n is an integer from 0-6; and
m is an integer from 0-6; or a -linker-reporter;
or a salt, tautomer, or optical isomer thereof.

9. The method of claim 7, wherein the Cleavable Terminating Moiety comprises a 2-nitrobenzyl substituent.

10. The method of claim 7, wherein the Primer is selected from oligonucleotides having a length between 8 to 100 nucleotides.

11. The method of claim 7, wherein the Base is selected from the group consisting of adenine, cytosine, guanine, thymine, uracil, modified pyrimidine and purine derivatives thereof, and mixtures thereof.

12. A composition for performing a multi-stage primer extension reaction comprising:
a) polynucleotide targets,
b) earlier stage primers capable of primer extension;
c) later stage primers comprising a photocleavable blocking group at 3′ ends, and
wherein the composition is in a vessel that is closed upon preparation of the composition.

13. The composition of claim 12, wherein the later stage primers are configured for unblocking by exposure to ultraviolet light.

14. The composition of claim 12, wherein the photocleavable blocking group has a blocking efficiency from about 90% to about 100%.

15. The composition of claim 12, comprising at least 5 pairs of target specific primers, alternatively at least 5 pairs of target specific primers, alternatively at least 10 pairs, or at least 20 pairs, or at least 50 pairs, or at least 100 pairs, or at least 200 pairs, or at least 500 pairs, or at least 1,000 pairs, or at least 2,000 pairs, or at least 5,000 pairs, or at least 10,000 pairs, or at least 20,000 pairs, of target specific primers.

16. The composition of claim 12, the earlier stage primers are present at a concentration of 0.01 to 0.5 μM, and the later stage primers are present at a concentration of 0.2 to 1 μM.

17. The composition of claim 16, wherein the later stage primers comprise a 3′ terminal nucleotide that is selected from the group consisting of:
5-[(S)-1-(5-methoxy-2-nitrophenyl)-2,2-dimethyl-propyloxy]methyl-2′-deoxy-uridine,
5-[(S)-1-(5-methoxy-2-nitrophenyl)-2,2-dimethyl-propyloxy]methyl-2′-deoxy-adenosine,
5-[(S)-1-(5-methoxy-2-nitrophenyl)-2,2-dimethyl-propyloxy]methyl-2′-deoxy-guanosine,
5-[(S)-1-(5-methoxy-2-nitrophenyl)-2,2-dimethyl-propyloxy]methyl-2′-deoxy-cytidine,
5-[(S)-1-(5-methoxy-2-nitrophenyl)-2,2-dimethyl-propyloxy]methyl-2′-deoxy-thymidine,
and mixtures thereof.

18. A method of preparing a photocleavable blocked primer comprising:
a) providing a primer precursor having a 3′ end; and
b) i) forming a duplex of the primer precursor hybridized to a template, wherein the template has a 5′ overhang relative to at least one nucleotide of the 3′ end of the primer precursor; and extending the primer precursor at its 3′ end by incorporating a nucleotide comprising a photocleavable blocking group with an DNA polymerase; or
ii) extending the primer precursor at its 3′ end by incorporating a nucleotide comprising a photocleavable blocking group with an template independent DNA polymerase.

19. The method of claim 18, wherein the nucleotide comprising a photocleavable blocking group is a compound of Formula II: 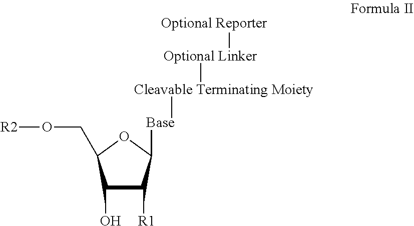
wherein:
R1 is H or OH;
R2 is H, monophosphate, diphosphate, triphosphate or α-thiotriphosphate;
Base is cytosine, uracil, thymine, adenine, or guanine, or modified pyrimidine and purine derivatives thereof;
Cleavable Terminating Moiety is a group imparting polymerase termination properties to the compound;
Optional Linker is a divalent group; and
Optional Reporter is a chemical moiety that is able to produce a detectable signal directly or indirectly.
